(19) United States
(12) Patent Application Publication
(10) Pub. No.: US 2013/0296688 A1
(43) Pub. Date: Nov. 7, 2013

(54) METHOD FOR EVALUATING A PET DATASET IN RELATION TO A NEUROTRANSMITTER AND / OR NEUROMODULATOR

(71) Applicant: SIEMENS AKTIENGESELLSCHAFT, Munich (DE)

(72) Inventors: BJÖRN HEISMANN, Erlangen (DE); Sebastian SCHMIDT, Weisendorf (DE)

(21) Appl. No.: 13/869,342

(22) Filed: Apr. 24, 2013

(30) Foreign Application Priority Data
May 3, 2012 (DE) 102012207315.9

(51) Int. Cl. A61B 5/00

(52) U.S. Cl. CPC A61B 5/0035
USPC 600/411

(57) ABSTRACT

A method is disclosed for evaluating a PET dataset with information concerning spatial distribution of a neurotransmitter and/or a neuromodulator in the brain of a patient wherein, in addition to the PET dataset, an especially morphometric magnetic resonance image dataset is recorded, which is or will be registered with the PET dataset. Concentration data of the spatial distribution is assigned to at least one specific region of the brain, especially to a nucleus, taking into account the magnetic resonance image data, for establishing result data assigned to the region.

### PRIORITY STATEMENT

The present application hereby claims priority under 35 U.S.C. §119 to German patent application number DE 102012207315.9 filed May 3, 2012, the entire contents of which are hereby incorporated herein by reference.

### FIELD

At least one embodiment of the invention generally relates to a method for evaluating a PET dataset with information concerning spatial distribution of a neurotransmitter and/or a neuromodulator in the brain of a patient and also to an assigned PET magnetic resonance device.

### BACKGROUND

Positron Emission Tomography (PET) is an imaging method already known in the prior art, in which a radio pharmaceutical (PET tracer) is administered to a patient. In such cases radio nuclides are used which emit positrons. If the PET tracer accumulates at specific locations and if a positron is emitted, two high-energy positrons are generated by the annihilation, which are sent out in exactly opposite directions and can be measured. Where an event has taken place can be deduced from coincidences.

In respect of neurological diseases, for example depressions, schizophrenia or Alzheimer's, the creation of PET datasets which describe the spatial distribution, thus ultimately the concentration of neurotransmitters or also neuromodulators in the human brain, is already known from the prior art. Since it is not possible because of the blood-brain barrier, to administer the substances to be detected directly in a marked variant, other ways are known for determining the distributions of neurotransmitters and/or neuromodulators in an indirect way or as a result of primary products. In the case of dopamine as a neurotransmitter in particular PET imaging has long been known, cf. for example Nora D. Volkow et al “PET-Evaluation of the Dopamine System of the Human Brain”, J. Nucl. Med 1996; 37: 1242-1256. Thus it is conceivable for example in relation to dopamine to mark a base product of the dopamine, for example Levadopa, with 11-C or 18-F. Despite this, methods have already also been proposed for other neurotransmitters, for example serotonin or acetylcholine, for measuring the spatial distribution within the brain.

In order to base a diagnosis on this type of PET data it is necessary to quantitatively relate the activity distributions of the PET measurement to areas of the patient's brain, which means that the measured presence of the neurotransmitter or neuromodulators must be related to specific areas of the brain (especially nuclei). For example one approach can be to determine the dopamine content in the striatum. However in this case there is the problem that only a coarse assignment to the area of the brain is possible, since corresponding anatomical information is not able to be obtained from the PET dataset. Thus it is not possible to determine the dopamine content in a specific area of the brain, especially a nucleus. If complementary computed tomography data is available, because of the restricted low-contrast resolution in the brain, this does not allow anatomical structures and areas of the brain to be segmented adequately well.

### SUMMARY

At least one embodiment of the invention specifies an option for the evaluation of PET data which is related to neurotransmitters and/or neuromodulators, which makes possible a highly-accurate quantified assignment, especially of concentrations, to specific areas of the brain.

In at least one embodiment, a method includes a provision for an especially morphometric magnetic resonance image dataset to be recorded in addition to the PET dataset, which is registered or will be registered with the PET dataset, wherein concentration data of the spatial distribution is assigned to at least one specific region of the brain, especially to a nucleus, taking into account the magnetic resonance image data for determining result data assigned to the region.

At least one embodiment of the invention hence proposes, in addition to the PET dataset of the brain, to also record a magnetic resonance image dataset of the brain, wherein especially advantageously sequences and protocols for morphometric images of the brain of the patient will be used. If the magnetic resonance image dataset and the PET dataset are then first registered to one another, the high-resolution magnetic resonance image data also showing the brain anatomically in outstanding quality is used to improve the assignment of PET data or of the corresponding concentration data to regions in the brain and thus especially to make possible a quantitative evaluation related to specific regions of the brain. Thus a computing device, for example as part of a control device, can determine from the PET dataset the spatial distribution of a neurotransmitter and/or of a neuromodulator, especially as concentration data and can assign this concentration data on the basis of the magnetic resonance image data to a specific brain region, especially a nucleus. Through a segmentation in the magnetic resonance data, the activity is finally related quantitatively to an area of the brain.

As well is the method, at least one embodiment of the invention also relates to a PET magnetic resonance device comprising a control device embodied for carrying out the inventive method. All information relating to at least one embodiment of the inventive method can similarly be transferred to the inventive PET magnetic resonance device, with which the same benefits can thus be obtained.

### BRIEF DESCRIPTION OF THE DRAWINGS

Further advantages and details of the present invention emerge from the example embodiments described below and also with reference to the drawings in which:

FIG. 1 shows a flowchart of an example embodiment of the inventive method,

FIG. 2 shows a sketch for the transmission of segmentations, and

FIG. 3 shows an inventive PET magnetic resonance device.

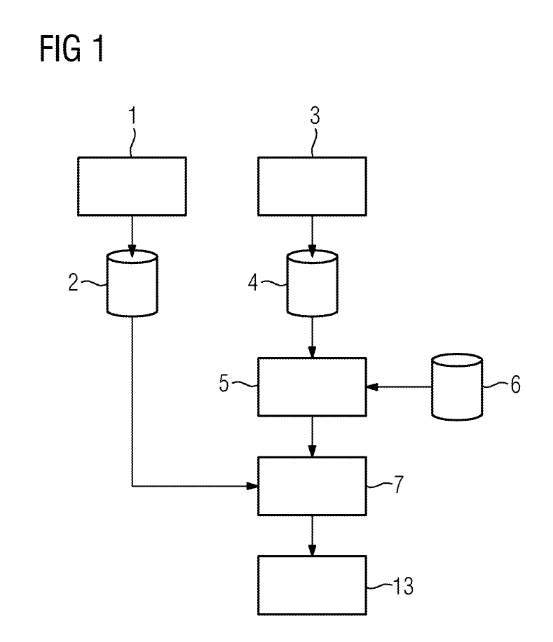

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

At least one embodiment of the invention hence proposes, in addition to the PET dataset of the brain, to also record a magnetic resonance image dataset of the brain, wherein especially advantageously sequences and protocols for morphometric images of the brain of the patient will be used. If the magnetic resonance image dataset and the PET dataset are then first registered to one another, the high-resolution magnetic resonance image data also showing the brain anatomically in outstanding quality is used to improve the assignment of PET data or of the corresponding concentration data to regions in the brain and thus especially to make possible a quantitative evaluation related to specific regions of the brain. Thus a computing device, for example as part of a control device, can determine from the PET dataset the spatial distribution of a neurotransmitter and/or of a neuromodulator, especially as concentration data and can assign this concentration data on the basis of the magnetic resonance image data to a specific brain region, especially a nucleus. Through a segmentation in the magnetic resonance data, the activity is finally related quantitatively to an area of the brain.

Hence, at least one embodiment of the inventive method allows quantitative region-related information to be derived for the first time from measurements of the distribution of neurotransmitters and/or neuromodulators marked with PET tracers, by taking account of segmentation information from a magnetic resonance image dataset. Hence neurotransmitter distributions and neuromodulator distributions in the brain of a patient can be measured in a quantitative and localized manner. This is of great interest for a plurality of widespread neurological diseases. For example dopamine imbalances, which play a large role in schizophrenia and delusions, can be verified in this way.

Finally PET data within specific regions is thus integrated so that the overall activity in a brain region can be determined. It should be noted in such cases that this is also meaningful longitudinally for comparative studies, for example in therapy control.

There is especially advantageously provision in such cases for the PET dataset and the magnetic resonance image dataset to be recorded with a combined PET magnetic resonance device. If a PET magnetic resonance device is used which has both a device for recording magnetic resonance data and also device for recording PET data, thus both modalities combined, the PET datasets and the magnetic resonance image datasets are principally registered with one another on the basis of the existing spatial correlation of the imaging device. Any possible movements of the patient can be actively spotted and corrected if necessary. In such cases however it should be noted that it is basically conceivable within the framework of at least one embodiment of the present invention to use different imaging devices and to use conventional registration methods for registering PET data and magnetic resonance image data.

In such cases, dopamine and/or serotonin and/or acetylcholine can be regarded as neurotransmitters for example, wherein other substances, especially also neuromodulators, are of course also conceivable. While neurotransmitters are to be understood as endogenic, biochemical neurotransmitters which pass on information between nerve cells, neuromodulators are chemical substances which exert influence on the operation of neurotransmitters. For marking the neurotransmitters and/or neuromodulators, C-11 and/or F-18 can be used as PET tracers for example. The striatum and/or the hippocampus and/or a prefrontal cortex region can be considered for example as regions of the brain, wherein other regions and nuclei are naturally also conceivable. Nuclei also refer as normal to an accumulation of nerve cell bodies within the central nervous system. In such cases these nuclear are usually surrounded by white matter and together with the cerebral cortex and cerebellar cortex form the grey matter.

In an especially advantageous embodiment of the present invention there can be provision for partial volume effects to be taken into account during the determination of the result data in the resolution of the magnetic resonance image dataset. In such cases, especially employing widely-used partial volume correction methods, ultimately the coarser resolution of the PET dataset is “computed down” to the far better resolution of the magnetic resonance image dataset, meaning that the larger voxels of the PET dataset, having edge lengths of 3 mm for example contain an item of measurement data which must be distributed to the very many smaller magnetic resonance voxels assigned to the PET voxel. If partial volume effects are taken into account a far better local resolution of the PET measurement and an improved assignment of the PET data to regions is produced.

In a further embodiment of the inventive method, there can be provision for the spatial distributions of a number of neurotransmitters and/or neuromodulators to be evaluated in respect of a differential diagnosis. It is thus conceivable to measure a number of substances, especially combined, and evaluate them using differential diagnosis, for example acetylcholine, dopamine and serotonin for depressions or Alzheimer's.

There can also be provision for the magnetic resonance image data to be used to carry out an attenuation correction for the PET data. Such attenuation corrections on the basis of magnetic resonance image data are already known in the prior art and can also be advantageously used within the framework of at least one embodiment of the present invention. Usually regions of the patient are segmented and the segmented regions are assigned attenuation values. For example in such cases an anatomical atlas, if necessary also already provided with attenuation values, can be taken into account. Regions not recorded in magnetic resonance imaging can be approximated in relation to the attenuation value, for example the arms and such like.

In an example first embodiment of the present invention, there can be provision for the regions in the magnetic resonance dataset to be determined by segmentation, especially explicitly predefined regions, especially nuclei, to be segmented, wherein the segmented regions are used by transmission of the PET dataset for the selection of PET data for evaluation. In such cases an anatomical atlas of the brain can expediently be used for the segmentation, in order to localize predefined regions. Preferably it is also already known beforehand the areas of the brain in which, especially the nuclei of the brain in which the PET tracers accumulate, or which areas are clinically relevant. If an anatomical magnetic resonance atlas is now used as the starting point for the segmentation, there can be provision for example for adapting the anatomical atlas by registration of the latter with the magnetic resonance image data for example, so that the precise localization and form of the relevant regions, especially nuclei, is recorded. Thus it is subsequently possible, in that the segmentation result for the regions to be evaluated is transmitted to the PET dataset, to select the PET data corresponding to the regions and to define for the regions the amount or concentration of the PET tracer and thus of the neurotransmitter or neuromodulator. Of course it is also especially advantageous in this context if partial volume effects are taken into account here.

It is especially advantageous in such cases for an atlas specific to at least one neurotransmitter and/or neuromodulator recorded in the PET data and/or annotated in respect of the latter to be used. Thus an anatomical atlas can be used which is specific to the respective neurotransmitter or neuromodulator, i.e. for example an anatomical atlas which includes specifically the dopamine-containing nuclei or general regions. Thus the dopaminergenic nuclei can be specifically segmented using this anatomical atlas and considered as regions within the framework of at least one embodiment of the present invention.

Preferably the atlas can comprise an expected spatial distribution of the neurotransmitter and/or neuromodulator. An atlas can thus be used which already contains information about the expected distribution of the neurotransmitters or neuromodulators, wherein they can be determined for example from prior examinations of healthy and or ill subjects. Literature data and/or neuropathological studies can also be taken into account. Hence it is then possible for example, within the framework of the evaluation to specify comparison values, for example relationships to expected result data and the like.

As an alternative, it is possible in this first embodiment of the inventive method, in which a segmentation in the magnetic resonance image dataset is transferred to the PET dataset, instead of using an anatomical atlas, for the regions also to be localized on the basis of at least one anatomical marker. In such cases anatomical landmarks within the brain are thus ultimately determined automatically and/or manually, with reference to which the position of regions of interest, especially nuclei, is produced so that these can then be assigned accordingly. For example landmarks can be considered here which have specific, also automatically recognizable forms and the like, so that corresponding algorithms can be used in order to make it possible to segment and identify regions in the brain as automatically as possible.

In a second alternative embodiment of the present invention, there can be provision for regions of high concentration in the PET dataset to be segmented, after which the segmented region is transferred into the magnetic resonance image dataset, wherein there edges corresponding to the segmented region of the PET data are determined and used for definition of the region in the magnetic resonance image dataset or, if no corresponding edges could be determined, the transmitted segmented region is defined directly as the region. In this variant of at least one embodiment of the invention, regions are thus initially segmented in the PET data which show a high activity of the PET tracer. The idea is now to use this segmentation of the PET data as the starting point of the segmentation in the magnetic resonance image data, wherein for each edge of the segmented regions, a corresponding signal jump in the (high-resolution) magnetic resonance image is sought. In this way it is then possible to define corresponding regions (and thus the regions of interest) in the magnetic resonance image dataset. If regions with different intensity are identified in the PET dataset to which no recognizable structures in the magnetic resonance image dataset correspond, the PET segmentation is directly transferred to the magnetic resonance image dataset in order to define a region of interest. Because of the high-resolution magnetic resonance image dataset it is thus possible to refine the segmentation in the PET dataset and especially also to obtain an assignment to specific regions, which in this embodiment can mostly be realized by visual appraisal. Especially taking into account partial volume effects however more precise data and assignments to specific regions are also to be obtained here. By contrast with the previously discussed first embodiment of the present invention, it is however not possible here or only possible with difficulty to establish whether there is no concentration or a concentration which is far too small in specific regions.

The segmentation in the PET data can be carried out on the basis of a threshold value-based segmentation method. It is additionally expedient in this context for a smoothing of segmented regions to be undertaken.

In general there can be provision for both the embodiments of the present invention for each magnetic resonance voxel of a region to be assigned a concentration value on the basis of the PET data, taking into account a partial volume correction. There is thus provision for calculating the concentration of the neurotransmitter and/or neuromodulator per magnetic resonance voxel in that, for each (larger) PET voxel, the amount or the concentration of the neurotransmitter and/or neuromodulator is defined and is assigned in the magnetic resonance image dataset to the magnetic resonance voxels of the region. Especially advantageously partial volume effects are once again taken into account in such cases in order to achieve an improved local assignment and accuracy of the definition. Thus ultimately a neurotransmitter amount or neuromodulator amount, i.e. a concentration, can be defined per magnetic resonance voxel.

In a development of embodiments of the inventive method, a global concentration of the neurotransmitter and/or of the neuromodulator can be determined, especially from a measurement of waste products in blood, liquor or urine and can be used for correction and/or formation of the relative value of determined concentration data and/or result data. If the concentration of neurotransmitters and/or neuromodulators or their waste products in blood, liquor or urine is defined, the global concentration of the neurotransmitter and/or neuromodulator in the organism can be defined and used for correction of the computed local concentration. The formation of relative values as result data is hence also possible. In-vitro markers are thus included in this embodiment.

There can further be provision for a table and/or graphic showing the concentration and/or the relative concentration of the neurotransmitter and/or the neuromodulator in different nuclei to be determined and displayed as result data. Thus neurotransmitter concentrations and/or neuromodulator concentrations (or amounts of matter) can be determined in different clinically-relevant regions, especially nuclei, and presented graphically or in tabular form. It is also possible for the neurotransmitter concentration and/or neuromodulator concentration of different nuclei to be related to one another and for these relationships to be presented, for example as a ratio of a dopamine concentration in the striatum and a dopamine concentration in the substantia nigra, which can represent an item of result data. A very wide range of basically known types of graphical result presentation are also conceivable in at least one embodiment of the present invention, or the color-coded overlaying or coloring-in of magnetic resonance image data as a function of concentrations and the like.

As well is the method, at least one embodiment of the invention also relates to a PET magnetic resonance device comprising a control device embodied for carrying out the inventive method. All information relating to at least one embodiment of the inventive method can similarly be transferred to the inventive PET magnetic resonance device, with which the same benefits can thus be obtained.

Such PET magnetic resonance devices, which ultimately combine two different imaging modalities with PET and magnetic resonance, are already known in the prior art. For example the PET detectors can be disposed within the patient chamber of a magnetic resonance device, surrounding said chamber. Naturally other embodiments are also conceivable. PET datasets and magnetic resonance image datasets of the brain, which are recorded with such a combined device, can now be evaluated immediately by the provision of the control devices embodied to carry out the inventive method and the result data can especially be obtained which can be determined entirely automatically by the control device.

The present invention will now be explained in greater detail with reference to a concrete example embodiment, in which dopamine, which has been marked with C-11 as PET tracer, is regarded as the neurotransmitter. A PET dataset shows in each voxel of the brain the activity of the PET tracer, which is a measure of the amount of dopamine contained in this voxel. This derived quantity data can be regarded in accordance with an embodiment of the invention as concentration data. The object is now to be able to assign the measurement results of the PET measurement as precisely as possible to specific nuclei or specific regions of the brain. In this example embodiment of the inventive method a PET magnetic resonance device is used, with which in parallel, i.e. ultimately at least partly overlapping in time, PET data and magnetic resonance image data of the brain can be recorded.

Initially in a step 1, a PET dataset 2 of the brain, as has been described, is recorded. In parallel hereto (or in another example embodiment at another time for a patient who has not moved) a magnetic resonance image dataset 4 of the brain is recorded in a step 3. The two datasets 2, 4 form the basis of the evaluation that now follows.

In this case in this example embodiment which corresponds to the first embodiment of the present invention, first of all in a step 5 regions of interest are segmented in the magnetic resonance image dataset 4. An anatomical atlas 6 is considered here, which is specifically designed for dopamine examinations, hence already explicitly contains the marked regions, specifically nuclei, in which a dopamine concentration is to be determined. Furthermore these predetermined regions which are to be segmented in the magnetic resonance image dataset 4, are already assigned the expected values for the dopamine concentration in the anatomical atlas 6.

There is now provision in step 5 for registering the anatomical atlas 6 with the magnetic resonance image data, so that the position of the predefined regions in the magnetic resonance image dataset 4 is known as the result of step 5.

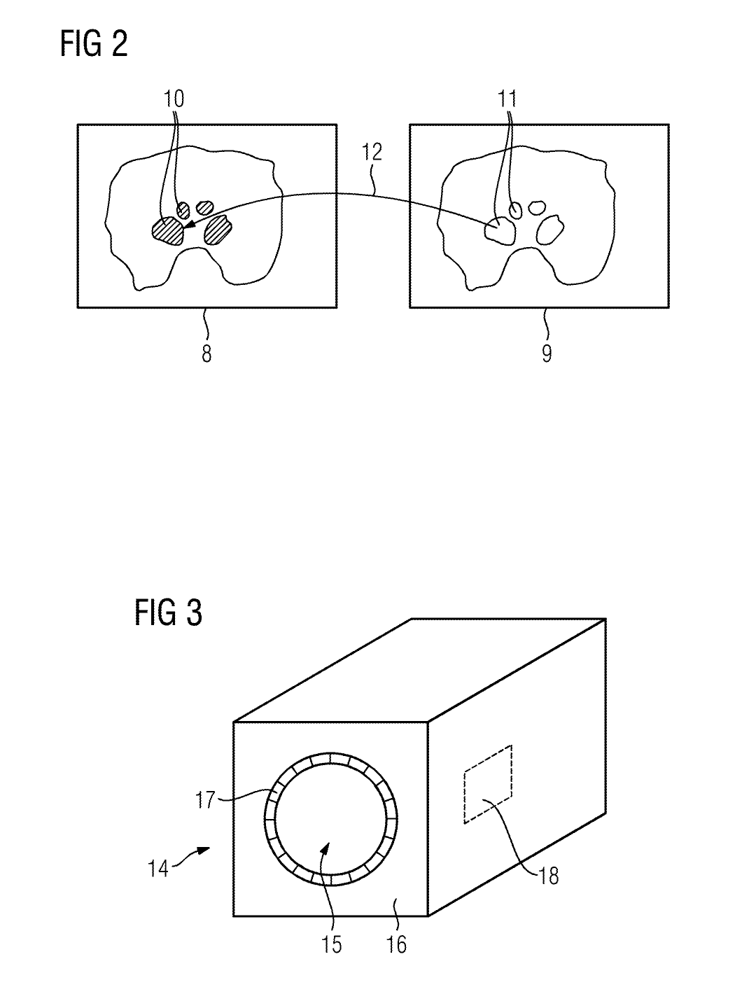

Since a combined PET-magnetic resonance device is used, the PET dataset 2 and the magnetic resonance image dataset 4 are registered to one another right from the outset. This means that the segmentation carried out in step 5 of regions of interest can be transferred to the PET dataset 2, meaning that smaller magnetic resonance voxels can be assigned to a corresponding larger PET voxels. This is done in step 7 and is to be explained in greater detail by the basic sketch in FIG. 2. Said figure shows schematic sections 8, 9 through the PET dataset 2 and the magnetic resonance image dataset 4 respectively. It is evident that more regions 10 exist in the PET dataset 2 (slice image 8) in which a higher concentration is present. In the magnetic resonance image dataset (slice image 9) prespecified regions are segmented and identified, for example the striatum, wherein the segmentation results 11 are shown there. These segmentation results 11 can now be transferred to the PET dataset 2, cf. arrow 12, so that a highly accurate assignment of the measured PET data in the areas 10 and the anatomical regions, especially nuclei, described by the segmentation results 11, is possible.

In step 7 each magnetic resonance voxel of a region is now assigned a concentration value on the basis of the PET data, wherein a partial volume correction is taken into account. The latter means that the different local resolution of the PET dataset 2 and of the magnetic resonance image dataset 4 are also taken into account. Thus partial volume contents are created for the magnetic resonance voxels. Finally a dopamine amount is determined as a concentration value for each magnetic resonance voxel, so that for each region a dopamine amount per magnetic resonance voxel, i.e. a concentration can be quantitatively determined.

In a step 13 suitable result data is determined from this, which can also directly be the determined concentrations. Naturally overall amounts of substance can also be established in the different clinically-relevant nuclei. It is also conceivable to relate the dopamine concentrations of different regions of interest, especially nuclei, to one another and to use these relationships as result data. The result data can be presented graphically or in tabular form. For example it is thus conceivable to determine and output as an item of result data the relationship between the dopamine concentration in the striatum and the dopamine concentration in the substantia nigra.

In this case it should also be noted at this point that it is also conceivable, if corresponding results or corresponding time is available, to include in-vitro markers in the computation of the result data, especially the concentration of dopamine or other waste products in blood, liquor or urine. Thus the global concentration of a dopamine in the organism can be determined and used for correction or relativization of the calculated local concentrations.

It should also be noted that the segmentation in step 5 can also be undertaken on the basis of anatomical landmarks (anatomical markers) if no suitable anatomical atlas 6 is available; the use of such an atlas is however preferred. In particular the expected values provided as annotations in the anatomical atlas 6 can namely also be used in order to generate corresponding result data.

While in the example embodiment presented here, the anatomical atlas 6 represents the starting point for the segmentation in the magnetic resonance image data, it is however also possible, in a second embodiment of the inventive method, to segment areas of high dopamine concentration in the PET dataset 2 and use them as a starting point for a segmentation in the magnetic resonance image dataset 4.

FIG. 3 finally shows a basic sketch of an inventive

PET-MR device 14.

This figure shows the PET detectors 17 provided surrounding the patient chamber 15 of a main magnetic field unit 16. It is thus possible to simultaneously record PET data and magnetic resonance image data.

The operation of the PET-magnetic resonance device 14 is controlled by a control device 18 which is embodied for carrying out embodiments of the inventive method.

Although the invention has been illustrated and described in greater detail by the preferred example embodiment, the invention is not restricted by the disclosed examples and other variations can be derived herefrom by the person skilled in the art without departing from the scope of protection of the invention.

What is claimed is:

1. A method for evaluating a PET dataset with information concerning spatial distribution of at least one of a neurotransmitter and a neuromodulator in the brain of a patient, the method comprising:
recording, in addition to the PET dataset, a morphometric magnetic resonance image dataset, which is or will be registered with the PET dataset, wherein concentration data of the spatial distribution is assigned to at least one specific region of the brain, taking into consideration magnetic resonance image data for determining result data assigned to the region.

2. The method of claim 1, wherein the PET dataset and the magnetic resonance image dataset are recorded with a combined PET-magnetic resonance device.

3. The method of claim 1, wherein at least one of
at least one of dopamine, serotonin, acetylcholine and noradrenalin are considered as neurotransmitters;
at least one of C-11 and 18-F-Levodopa are used as PET tracers; and
at least one of the striatum, the hippocampus and a prefrontal cortex region are considered as the region of the brain.

4. The method of claim 1, wherein partial volume effects are taken into account in a determination of the result data in the resolution of the magnetic resonance image dataset.

5. The method of claim 1, wherein the spatial distributions of a number of at least one of the neurotransmitters and neuromodulators are evaluated in respect of a differential diagnosis.

6. The method of claim 1, wherein the magnetic resonance image data is used for carrying out an attenuation correction for the PET data.

7. The method of claim 1, wherein the regions in the magnetic resonance image dataset are determined by segmentation, and wherein the segmented regions are used for selection of PET data for evaluation by transmission to the PET dataset.

8. The method of claim 7, wherein the regions are especially explicitly predefined regions, and wherein an anatomical atlas of the brain is used during the segmentation in order to localize the predefined regions.

9. The method of claim 8, wherein an atlas specific to at least one of at least one neurotransmitter and neuromodulator, at least one of recorded in the PET data and annotated in relation thereto, is used.

10. The method of claim 9, wherein the atlas comprises an expected spatial distribution of at least one of the neurotransmitter and neuromodulator.

11. The method of claim 7, wherein the regions are localized on the basis of at least one anatomical marker.

12. The method of claim 1, wherein at least one region of high concentration is segmented in the PET data, after which the segmented region is transmitted into the magnetic resonance image dataset wherein edges corresponding to the segmented region of the PET data are determined and used for the definition of a region in the magnetic resonance image dataset or, if no corresponding edges are determined, the transmitted segmented region is used directly as the region.

13. The method of claim 12, wherein at least one of
the segmentation is carried out on the basis of a threshold value-based segmentation method, and
a smoothing of segmented regions is carried out.

14. The method of claim 7, wherein each magnetic resonance voxel of a region is assigned a concentration value based on the PET data.

15. The method of claim 1, wherein a global concentration of at least one of the neurotransmitter and the neuromodulator is determined and is used for at least one of correction and formation of a relative value of at least one of determined concentration data and result data.

16. The method of claim 1, wherein at least one of a table and graphic presenting at least one of the concentration and the relative concentration of at least one of the neurotransmitter and of the neuromodulator in different nuclei is determined and displayed as result data.

17. A PET-magnetic resonance device, comprising:
a control device, embodied to carry out the method of claim 1.

18. The method of claim 1, wherein the at least one specific region of the brain includes a nucleus.

19. The method of claim 2, wherein at least one of at least one of dopamine, serotonin, acetylcholine and noradrenalin are considered as neurotransmitters;
at least one of C-11 and 18-F-Levodopa are used as PET tracers; and
at least one of the striatum, the hippocampus and a prefrontal cortex region are considered as the region of the brain.

20. The method of claim 7, wherein the regions are especially explicitly predefined regions, and wherein nuclei are segmented.

21. The method of claim 14, wherein each magnetic resonance voxel of a region is assigned a concentration value based on the PET data, taking into account a partial volume correction.

22. The method of claim 15, wherein a global concentration of at least one of the neurotransmitter and the neuromodulator is determined from a measurement of waste products in blood, liquor or urine.

23. A PET-magnetic resonance device, comprising:
a control device, embodied to carry out the method of claim 7.
